(19) United States
(12) Patent Application Publication
(10) Pub. No.: US 2016/0175577 A1
(43) Pub. Date: Jun. 23, 2016

(54) Ureteral Catheter Connector Adapter

(71) Applicant: Anthony Solazzo, South Plainfield, NJ (US)

(72) Inventor: Anthony Solazzo, South Plainfield, NJ (US)

(21) Appl. No.: 14/578,759

(22) Filed: Dec. 22, 2014

(51) Int. Cl. A61M 39/10; A61M 25/00

(52) U.S. Cl. CPC A61M 39/10; A61M 25/0097; A61M 2025/0004; A61M 2039/1077

(57) ABSTRACT

The kit includes an adapter that improves the fixation of a ureteral catheter to the adapter and a clip for stabilizing the combining of ureteral catheters with a Foley catheter. A collar at each ureteral catheter-receiving orifice of the adapter increases the length over which a ureteral catheter is connected to the adapter thereby reducing the risk of the ureteral catheter pulling out during a surgical procedure. A cap is tethered to the adapter to close off and seal an orifice should only one ureteral catheter be used.

This invention relates to a ureteral catheter connector adapter. More particularly, this invention relates to a connector adapter for interconnecting a Foley catheter or other type of catheter with ureteral catheters so that they may drain into the same drainage bag.

As is known, for example, as disclosed in U.S. Pat. No. 5,531,723, during the course of various intraoperative procedures, a urologist may be called upon to assist general surgeons or other specialists for the placement of ureteral catheters in the ureters for their easier identification during difficult abdominal or pelvic surgery. Once these are in place, a Foley catheter is inserted into the bladder for fixation of the ureteral catheters so that they do not migrate out.

In order to facilitate the collection of fluids from the Foley catheter and the ureteral catheters, U.S. Pat. No. 5,531,723 describes various adapters that may be used to connect a Foley catheter and one or two ureteral catheters together for delivery of fluids to a common drainage bag.

It is an object of the invention to improve the fixation of a ureteral catheter to an adapter.

It is another object of the invention to stabilize the combining of ureteral catheters with a Foley catheter.

Briefly, the invention provides a one-piece adapter that receives a Foley catheter as well as one or two ureteral catheters for delivery of fluids to a common collection device.

The adapter is made of a suitable biocompatible material, such as a plastic material, and comprises a main body portion and a shoulder portion.

The main body portion defines a main flow path for conducting a flow of fluid from a Foley catheter connected to an inlet end of the flow path to the collection device, e.g. a drainage bag connected to an outlet end of the flow path.

The shoulder portion is located at an inlet end of the main body portion and has a pair of orifices therein and a pair of collars thereon. Each orifice is in communication with the main flow path and each collar is disposed at an inlet end of a respective orifice for receiving a ureteral catheter for delivery of a flow of fluid therethrough to the main flow path. The inclusion of the collar increases the length over which a ureteral catheter is connected to the adapter, thereby, reducing the risk of the ureteral catheter pulling out of the adapter during a surgical procedure.

The adapter is also provided with a means for sealing off an orifice of the adapter should there be only one ureteral catheter involved in the procedure. In this regard, with one ureteral catheter inserted in the adapter to communicate with an orifice therein, the other orifice can be sealed to prevent leakage.

In one embodiment, the means for sealing off one orifice of the adapter is in the form of a cap that is integrally tethered to the shoulder portion for movement between an open position spaced from one of the collars and a closure position in the collar to prevent entry of a ureteral catheter therein and to close the orifice associated therewith.

The invention also provides a clip for engagement with a Foley catheter and at least one ureteral catheter in order to stabilize the combining of ureteral catheters with a Foley catheter.

The clip is sized to be closed about a Foley catheter and one or two ureteral catheters in order to hold the catheters together as a unit rather than having the catheters arranged loosely relative to each other during a surgical procedure.

The clip comprises a pair of hinged arcuate sections for receiving a Foley catheter therebetween when the clip is closed. In addition, each arcuate section is formed with a recess for receiving a ureteral catheter when the clip is closed.

The clip also has a pair of tabs to permit manual biasing of the arcuate sections away from each other in response to squeezing of the tabs towards each other as well as a locking arrangement at the free ends of the arcuate sections to lock the arcuate sections in the closed position.

The invention also provides a kit for combining a Foley catheter and at least one ureteral catheter comprised of an adapter, as above, and a clip, as above.

Likewise, the invention provides a system comprised of a Foley-catheter, at least one ureteral catheter, an adapter, as above, a clip, as above, and a collection device.

These and other objects and advantages will become more apparent from the following detailed description taken in conjunction with the accompanying drawings wherein:

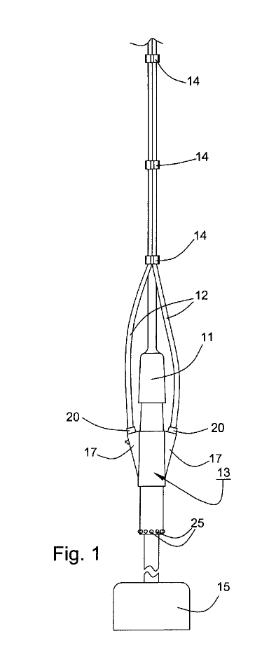

FIG. 1 illustrates a system in accordance with the invention;

FIG. 2 illustrates a front view of an adapter employed in the system of FIG. 1;

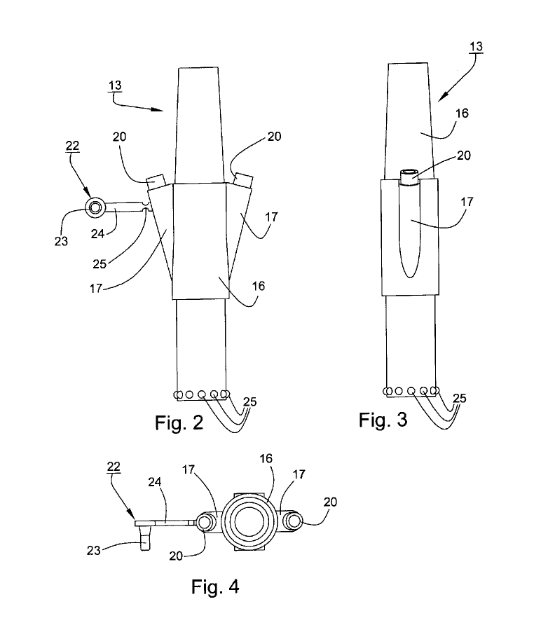

FIG. 3 illustrates a side view of the adapter of FIG. 2;

FIG. 4 illustrates a top view of the adapter of FIG. 2;

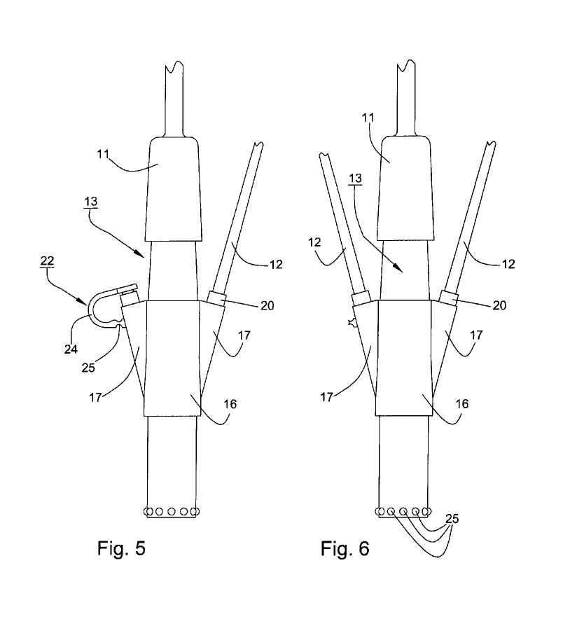

FIG. 5 illustrates a front view of the adapter of FIGS. 2 to 4 with one ureteral catheter in place and one orifice closed in accordance with the invention;

FIG. 6 illustrates a front view of the adapter of FIGS. 2 to 4 with two ureteral catheters in place;

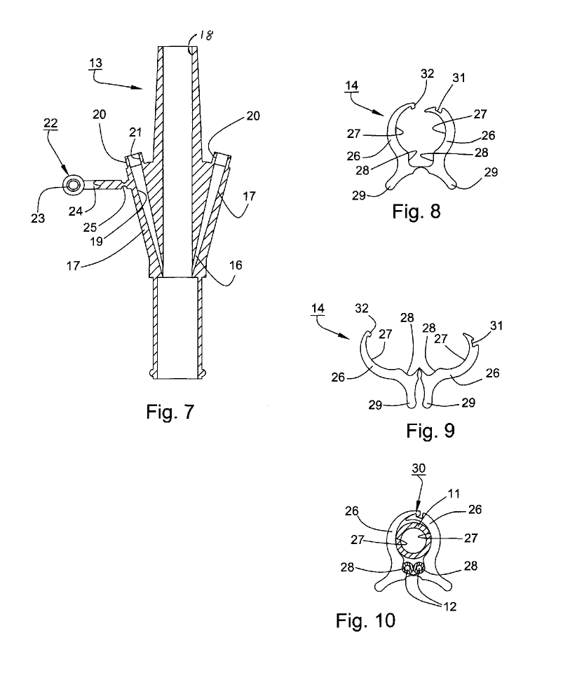

FIG. 7 illustrates a cross-sectional view of the adapter of FIGS. 2 to 4;

FIG. 8 illustrates a view of a clip employed in the system of FIG. 1;

FIG. 9 illustrates a view of the clip of FIG. 8 in a fully open position; and

FIG. 10 illustrates a cross-sectional view of the clip of FIG. 8 in a closed position about a Foley catheter and a pair of ureteral catheters in accordance with the invention;

Referring to FIG. 1, the system comprises a Foley catheter 11, a pair of ureteral catheters 12, an adapter 13, a plurality of clips 14 and a collection device 15 in the form of a drainage bag.

The Foley catheter 11, ureteral catheters 12 and collection device 15 are of conventional structure and need not be further described.

Referring to FIGS. 2 to 4, the adapter 13 is made of one piece with a main body portion 16 and a shoulder portion 17 similar to that as described in U.S. Pat. No. 5,531,723.

As illustrated in FIG. 7, the main body portion 16 defines a main flow path 18 in communication with the Foley catheter 11 (see FIG. 1) for conducting a flow of fluid from the Foley catheter 11 to the collection device 15 (see FIG. 1). The shoulder portion 17 is located at an inlet end of the main body portion 16 and has a pair of orifices 19, each of which is in communication with the main flow path 18.

Referring to FIGS. 2 to 4 and 7, the shoulder portion 17 also has a pair of collars 20, each of which is disposed at an inlet end of a respective orifice 19 (see FIG. 7). As illustrated in FIG. 1, each collar 20 is sized to receive a ureteral catheter 12 therein for delivery of a flow of fluid therethrough to the main flow path 18.

As shown in FIG. 7, each orifice 19 is tapered to accommodate different sizes of ureteral catheters 12. However, each collar 20 has a bore 21 of uniform diameter equal to the diameter at the inlet end of an orifice 19.

The inclusion of the collar 20 increases the length over which a ureteral catheter 12 is connected to the adapter 13, thereby reducing the risk of the ureteral catheter 12 pulling out of the adapter during a surgical procedure.

Referring to FIGS. 2 to 5 and 7, the adapter 13 is also provided with a means 22 for sealing off an orifice 19 of the adapter 13 should there be only one ureteral catheter 12 involved in a surgical procedure. In this regard, with one ureteral catheter 12 inserted in the adapter 13 to communicate with an orifice 19 therein, the other orifice 19 can be sealed to prevent leakage as indicated in FIG. 5.

In one embodiment, the means 22 for sealing off one orifice of the adapter 13 is in the form of a cap 23 that is integrally connected to the shoulder portion 17 by a tether 24 for movement between a closure position in one of the collars 20 to prevent entry of a ureteral catheter 12 therein and to close the orifice disposed therewith (see FIG. 5) and an open position spaced from the collar 20 (see FIG. 2).

As indicated in FIG. 7, the tether 24 has a tear-away notch 25 located adjacent the shoulder portion 17 to allow for removal of the cap 23 and tether 24 (see FIG. 6) should there be no need to close off a ureteral catheter receiving orifice 19 during a surgical procedure. Thus, the cap 23 and tether 24 in a position as indicated in FIG. 2 need not interfere with a surgical procedure.

Referring to FIGS. 2 and 3, the adapter 13 is also provided with a plurality of circumferentially disposed projections 25 on an exterior proximal surface of the main body portion 16 to provide a manual gripping surface.

Referring to FIGS. 8 to 10, each clip 14 comprises a pair of arcuate sections 26 made of a resilient plastic and disposed in hinged relation to pivot between an open position relative to each other, as shown in FIG. 9, and a closed position relative to each other, as shown in FIG. 10.

Each arcuate section 26 has a curvilinear portion 27 defining a first transverse passage with the other arcuate section 26 for receiving the Foley catheter 11 therein when in the closed position (FIG. 10).

Each arcuate section 26 also has a recess 28 defining a second transverse passage for receiving a ureteral catheter 12 therein when in the closed position (FIG. 10).

Each clip 14 also has a pair of tabs 29 to permit manual biasing of the arcuate sections 26 away from each other in response to squeezing of the tabs 29 towards each other. As illustrated, each tab 29 is integral with and extends outwardly of one end of a respective arcuate section 26.

Each clip 14 also has a locking arrangement 30 at the free ends to lock the arcuate sections 26 in the closed position. As illustrated, the locking arrangement 30 includes a recess 31 in a free end of one arcuate section 26 and a tang 32 at the free end of the other arcuate section 26 for engaging in the recess 31 with the arcuate sections 26 in the closed position of FIG. 10 to lock the arcuate sections 26 in the closed position.

As indicated in FIG. 10, when a clip 14 is closed about the Foley catheter 11 and a pair of ureteral catheters 12, the catheters 11, 12 are snuggly received so as to be held together. As indicated in FIG. 1, multiple clips 14 may be used to combine the ureteral catheters 12 with the Foley catheter 11.

The invention thus provides a kit that includes an adapter that improves the fixation of a ureteral catheter to the adapter and a clip for stabilizing the combining of ureteral catheters with a Foley catheter.

What is claimed is:

1. A one-piece adapter comprising
a main body portion defining a main flow path for conducting a flow of fluid from a Foley catheter connected to an inlet end of said flow path to a drainage bag connected to an outlet end of said flow path; and
a shoulder portion at an inlet end of said main body portion having a pair of orifices therein and a pair of collars thereon, each said orifice being in communication with said main flow path and each said collar being disposed at an inlet end of a respective orifice of said pair of orifices for receiving a ureteral catheter therein for delivery of a flow of fluid therethrough to said main flow path.

2. A one-piece adapter as set forth in claim 1 further comprising a cap connected to said shoulder portion for movement between an open position spaced from one of said pair of collars and a closure position in said one of said pair of collars to prevent entry of a ureteral catheter therein and to close said orifice associated therewith to the flow of fluid.

3. A one-piece adapter as set forth in claim 2 further comprising a tether connecting said cap to said shoulder portion, said tether having a tear-away notch therein adjacent said shoulder portion to allow for removal of said cap and tether from said shoulder portion.

4. A one-piece adapter as set forth in claim 1 further comprising a plurality of circumferentially disposed projections on an exterior proximal surface of said main body portion.

5. A clip for engagement with a Foley catheter and at least one ureteral catheter, said clip comprising
a pair of arcuate sections disposed in hinged relation to pivot between an open position relative to each other and a closed position relative to each other, each said arcuate section having a curvilinear portion defining a first transverse passage with the other of said arcuate sections for receiving a Foley catheter therein and a recess defining a second transverse passage for receiving a ureteral catheter therein.

6. A clip as set forth in claim 5 wherein one of said arcuate sections has a recess at a free end thereof and the other of said arcuate sections has a tang at a free end hereof for engaging in said recess with said arcuate sections in said closed position to lock said arcuate sections in said closed position.

7. A clip as set forth in claim 6 wherein said arcuate sections are made of a resilient plastic.

8. A clip as set forth in claim 5 further comprising a pair of tabs, each said tab being integral with and extending outwardly of one end of a respective arcuate section to permit manual biasing of said arcuate sections away from each other in response to squeezing of said tabs towards each other.

9. A kit for combining a Foley catheter and at least one ureteral catheter comprising
a one-piece adapter comprising a main body portion defining a main flow path for conducting a flow of fluid from a Foley catheter connected to an inlet end of said flow path to a drainage bag connected to an outlet end of said flow path and a shoulder portion at an inlet end of said main body portion having a pair of orifices therein, each said orifice being in communication with said main flow path for delivery of a flow of fluid therethrough to said main flow path; and
a clip comprising a pair of arcuate sections disposed in hinged relation to pivot between an open position relative to each other and a closed position relative to each other, each said arcuate section having a curvilinear portion defining a first transverse passage with the other of said arcuate sections for receiving a Foley catheter therein and a recess defining a second transverse passage for receiving a ureteral catheter therein.

10. A kit as set forth in claim 9 wherein said shoulder portion includes a pair of collars thereon, each said collar being disposed at an inlet end of a respective orifice of said pair of orifices for receiving a ureteral catheter therein for delivery of a flow of fluid therethrough to said main flow path.

11. A kit as set forth in claim 10 wherein said adapter further comprises a cap connected to said shoulder portion for movement between a closure position in one of said pair of collars to prevent entry of a ureteral catheter therein and to close said orifice disposed therewith to the flow of fluid and an open position spaced from said one collar.

12. A kit as set forth in claim 11 further comprising a tether connecting said cap to said shoulder portion, said tether having a tear-away notch therein adjacent said shoulder portion to allow for removal of said cap and tether from said shoulder portion.

13. A kit as set forth in claim 9 wherein said adapter further comprises a plurality of circumferentially disposed projections on an exterior proximal surface of said main body portion.

14. A system comprising
a Foley-catheter;
at least one ureteral catheter;
an adapter having a main body portion defining a main flow path in communication with said Foley catheter for conducting a flow of fluid from said Foley catheter to said main flow path and a shoulder portion at an inlet end of said main body portion having a pair of orifices therein, each said orifice being in communication with said main flow path with one of said pair of orifices being in communication with said ureteral catheter for delivery of a flow of fluid therethrough to said main flow path;
a clip comprising a pair of arcuate sections disposed in hinged relation to pivot between an open position relative to each other and a closed position relative to each other, each said arcuate section having a curvilinear portion defining a first transverse passage with the other of said arcuate sections and receiving said Foley catheter therein and at least one of said arcuate sections having a recess defining a second transverse passage receiving said ureteral catheter therein; and
a collection device in communication with an outlet of said main flow path to receive fluid therefrom.
